(19) United States
(12) Patent Application Publication
(10) Pub. No.: US 2016/0194263 A1
(43) Pub. Date: Jul. 7, 2016

(54) Method for Producing N-Butane Derivatives

(71) Applicant: OXEA GmbH, Oberhausen (DE)

(72) Inventors: Matthias Eisenacher, Huenxe (DE); Heinz Strutz, Moers (DE)

(21) Appl. No.: 14/771,521

(22) PCT Filed: Jun. 25, 2014
(86) PCT No.: PCT/EP2014/063345

§ 371 Date: Aug. 31, 2015

(30) Foreign Application Priority Data
Jun. 28, 2013 (DE) 10 2013 106 787.5

(51) Int. Cl. C07C 29/34; C07C 45/45; C07C 45/32; C07C 29/141; C07C 67/40; C07C 51/235; C07C 209/24; C07C 41/09; C07C 29/14; C07C 29/149; C07C 45/62

(52) U.S. Cl. CPC C07C 29/34; C07C 29/149; C07C 45/45; C07C 45/32; C07C 29/141; C07C 45/62; C07C 51/235; C07C 209/24; C07C 41/09; C07C 29/14; C07C 67/40

(57) ABSTRACT

The present invention relates to a method of synthesizing n-butane derivatives, especially 1-butanol, 1-butanal or 1-butyric acid as well as crotonaldehyde and the reaction products thereof, wherein at first methanol is converted into a C₂ building block, either ethanol or acetaldehyde, and said C₂ building block is then dimerized into a C₄ building block.

### CLAIM FOR PRIORITY

This application is a national phase application of PCT/EP2014/063345 FILED Jun. 25, 2014 which was based on application DE 10 2013 106 787.5 FILED Jun. 28, 2013. The priorities of PCT/EP2014/063345 and DE 10 2013 106 787.5 are hereby claimed and their disclosures incorporated herein by reference.

### TECHNICAL FIELD

The present invention relates to a method for the synthesis of n-butane derivatives, including n-butanol, n-butanal and n-butyric acid, n-butylamines as well as butyl acetate, 2-ethyl hexanol and 2-ethylhexanoic acid as well as compounds that can be derived from croton aldehyde, particularly sorbic acid, 3-methoxy butanol and crotonic acid.

### BACKGROUND

N-Butanol is an important industrial organic intermediate, which as it is or after derivatization finds use in a variety of applications, for example, in butyl esters such as butyl acetate, for example, as a solvent in the paint and coating sector.

N-butyraldehyde has great economic importance due to the aldehyde activity, thus, for example, the product range of primary, secondary and tertiary butylamines is accessible by reductive amination. By use of dimerization and reduction 2-ethylhexanol is obtained, which has gained enormous economic importance as a plasticizer alcohol; the oxidation product 2-ethylhexanoic acid is also an important monocarboxylic acid for the production of esters used as plasticizers and lubricants. Butyric acid is also an important industrial chemical.

Due to the active double bond and the aldehyde group croton aldehyde finds use in various applications, for example, for the production of sorbic acid which is used as a preservative in the food industry, for the production of 3-methoxybutanol, which is used as a hydraulic fluid or in the form of acetate ester as solvent for paints or for the production of crotonic acid, which is used as a comonomer in polymerization reactions.

The synthesis of n-butane derivatives conventionally starts from propene, which is then converted in an oxo or hydroformylation reaction into a mixture of n- and isobutyraldehyde. The oxo reaction immanently involves a potential discrepancy between the yield of isobutyraldehyde and its demand in the market. This discrepancy results in an apparent desire for selective accesses to the quantitatively more significant n-C4 aldehyde, which provides a more universal industrial downstream chemistry.

Because of the immense importance of n-butane derivatives for the technical organic chemistry, thus, there is a constant need for improvements and novel synthesis routes.

### SUMMARY OF INVENTION

Thus, it is an object to provide a synthesis route of n-butane derivatives, which in particular dispenses with the use of propene as a reactant. This object is achieved by the method according to the invention. Accordingly it is provided a method for the synthesis of n-butane derivatives, comprising the steps of:
a) conversion of methanol into a C₂ building block
b) dimerization of the C₂ building block into a C₄ building block
c) optionally further conversions of the thus obtained C₄ building block.

Thus, the synthesis process enables a synthesis of n-butane derivatives starting from readily available C₁ building blocks such as methanol. The method according to the invention offers for most applications in particular one or more of the following advantages:
Because n-butane derivatives are synthesized by means of an initial reaction of two C₁ building blocks, wherein one of said building blocks is methanol, the use of propene can be dispensed with.
The raw material methanol is available in large quantities and with low costs on the basis of different sources of raw materials such as natural gas, coal or biomass and can readily transported as a liquid compound at normal pressure and room temperature.
The resulting n-butane derivatives are highly isomerically pure and can therefore be converted into products, which also have a higher purity (such as the already mentioned 2-ethylhexanol). The complex separation processes of n- and isobutane derivatives required within the framework of the propene-based oxo-chemistry can be dispensed with. The reaction pressures which are typical in the oxo-chemistry and are associated with an extensive technical equipment can be dispensed with.

### DETAILED DESCRIPTION

“n-butane derivatives” in the sense of the present invention are in particular 1-butanol, 1-butanal, 1-butyric acid, 1-butylamine, 2-ethylhexanol, 2-ethylhexanoic acid, butyl acetate, crotonaldehyde, sorbic acid, crotonic acid, 3-methoxy butanol and mixtures thereof.

In the following the individual steps of the method are explained.

a) Conversion of methanol into a C₂ building block

As C₂ building blocks, which are well suited for the subsequent dimerization, in particular ethanol and acetaldehyde come into consideration; these, thus, each represent preferred embodiments of the present invention.

1) Conversion into Ethanol

In a first preferred embodiment at first methanol is converted into acetic acid. One possible conversion is, inter alia, described in “Ullmann's Encyclopedia of Industrial Chemistry”, Wiley-VCH, 6th ed. 2003, vol. 1, pages 151-165. Here, methanol is carbonylated with carbon monoxide in the presence of a rhodium or iridium catalyst.

The ethanol synthesis may then be conducted such that, for example, according to Arpe “Industrielle Organische Chemie”, Wiley-VCH, 6th ed., p. 198, at first acetic acid with further methanol is converted into methyl acetate, which by means of gas phase hydrogenolysis is cleaved into ethanol and methanol; the methanol obtained can, of course, be returned either for a new esterification or for the acetic acid synthesis.

Alternatively, as described, for example, in WO 2011/056595 or WO 2011/056597, the acetic acid can be hydrogenated into ethanol with hydrogen and an appropriate catalyst.

Alternatively, methanol can also be homologized directly into ethanol by means of CO/H₂. For this purpose, iron-cobalt carbonyls with the addition of iodide promoters at temperatures of 100 to 250° C. and pressures of 5 to 100 MPa have been proven effective, as described, inter alia, in “Ullmann's Encyclopedia of Industrial Chemistry”, Wiley-VCH, 6th ed. 2003, vol. 12, pages 404-405, and/or U.S. Pat. No. 4,320,230.

### 2) Conversion Into Acetaldehyde

Acetaldehyde can be derived from ethanol (which can be represented as described above) by means of oxidation. The oxidation of ethanol is preferably carried out by passing an ethanol-air mixture at 500-650° C. over a silver catalyst or by means of dehydrogenation in the gas phase at 260-290° C. at promoted copper catalysts. In this respect it is exemplarily referred to “Ullmann's Encyclopedia of Industrial Chemistry”, Wiley-VCH, 6th ed. 2003, vol. 1, pages 135-136.

The above-described methanol homologation into ethanol can—with modified reaction conditions, in particular a modified CO/H₂ ratio, temperature and/or pressure—also be used for the direct synthesis of acetaldehyde.

Alternatively, the acetic acid described in step 1) can be reduced to acetaldehyde, as described, for example, in WO 2010/014146 A2.

b) Dimerization of the C₂ building block into a C₄ building block

### 1) Dimerization Starting From Ethanol

Ethanol can be dimerized directly into butanol in the sense of a Guerbet reaction. Preferred reaction conditions are, inter alia, described in Kirk-Othmer, Encyclopedia of Chemical Technology, 3rd ed., Wiley-lntersciences, New York, 1980, p. 372 and/or Journal of Organic Chemistry, vol. 22, 1956, pages 540-542.

### 2) Dimerization Starting From Acetaldehyde

In a dimerization starting from acetaldehyde it is preferable at first dimerized into crotonaldehyde (=2-butenal). Preferred reaction conditions are described, inter alia, in “Ullmann's Encyclopedia of Industrial Chemistry”, Wiley-VCH, 6th ed. 2003, vol. 9, pages 702-703 and/or DE 349915 C. The crotonaldehyde thus obtained then can be converted either into butanol or (if only the alkene functionality is reduced) into butyraldehyde.

For the reduction to butanol, among others, copper or nickel catalysts can be used. Preferred reaction conditions are described, inter alia, in “Ullmann's Encyclopedia of Industrial Chemistry”, Wiley-VCH, 6th ed. 2003, vol. 5, pages 717-718 and/or DE 33801 C.

For the reduction to butyraldehyde crotonaldehyde can be hydrogenated in the gas or liquid phase at a copper, nickel or palladium catalyst. Preferred reaction conditions are described, inter alia, in “Ullmann's Encyclopedia of Industrial Chemistry”, Wiley-VCH, 6th ed. 2003, vol. 5, page 696 and/or DE 540327 C.

c) Optional further conversions

The butyraldehyde obtained in step b) or the butanol may be oxidized into butyric acid or reductively aminated into 1-butylamines (Ullmann's Encyclopedia of Industrial Chemistry, Wiley-VCH, 6th ed. 2003, vol. 6, page 501; vol. 2, pages 383-384).

The butanol obtained in step b) can also be oxidized into butyraldehyde. To this end, inter alia, copper-based catalysts are used. Preferred reaction conditions are described, inter alia, in “Ullmann's Encyclopedia of Industrial Chemistry”, Wiley-VCH, 6th ed. 2003, vol. 5, page 696 and/or DE 832292 C.

If butyraldehyde has been produced in step b), it can alternatively be reduced to butanol.

The butyraldehyde obtained in step b) or c) can be used for further aldehyde typical conversions; here, in particular the production of 2-ethylhexanol via aldol condensation and complete hydrogenation or of 2-ethylhexanoic acid via aldol condensation, partial hydrogenation and oxidation of the intermediate 2-ethylhexanals should be mentioned.

The butanol obtained in step b) or c) can in particular be further converted into n-butyl acetate (see Arpe “Industrielle Organische Chemie”, Wiley-VCH, 6th ed. p. 197).

The crotonaldehyde produced in step b) in addition to the production of butyraldehyd and butanol can be used as an intermediate for the production of crotonic acid, methoxybutanol and sorbic acid. Preferred reaction conditions are described, inter alia, in Arpe “Industrielle Organische Chemie”, Wiley-VCH, 6th ed., pages 204-205.

The above-mentioned components as well as the components to be used according to the invention which are claimed and described in the exemplary embodiments are not subject to particular conditions of exemption with respect to their size, shape, material selection and technical concept, such that the selection criteria known in the field of application can be applied without limitation.

The individual combinations of the constituents and of the features of the above-mentioned embodiments are exemplary, and the replacement and the substitution of these teachings with other teachings that are contained in this document with the references cited are also explicitly contemplated. The person skilled in the art will recognize that variations, modifications and other embodiments that are described herein, may also occur without departing from the spirit and scope of the invention. Accordingly, the above description is illustrative and not to be considered as limiting. The term “include” or “comprise” used in the claims does not exclude other components or steps. The indefinite article “a” does not exclude the meaning of a plural. The mere fact that certain measures are cited in mutually different claims, does not mean that a combination of these measures could not be used advantageously. The scope of the invention is defined in the following claims and the associated equivalents.

1. Method for the synthesis of n-butane derivatives, including the steps of:
a) conversion of methanol into a C₂ building block;
b) dimerization of the C₂ building block into a C₄ building block;
c) optionally further conversions of the C₂ building block thus obtained.

2. Method according to claim 1, wherein the C₂ building block is ethanol.

3. Method according to claim 1, wherein the C₂ building block is acetaldehyde.

4. Method according to claim 1, wherein in step a) at first in a step a1) methanol is converted into acetic acid, which is then reduced in a step a2).

5. Method according to claim 1, wherein at first methanol is converted into ethanol by reaction with a synthesis gas.

6. Method according to claim 1, wherein methanol is converted into acetaldehyde by reaction with a synthesis gas.

7. Method according to claim 1, wherein acetaldehyde is formed by oxidation or dehydrogenation of ethanol.

8. Method according to claim 1, wherein in step b) ethanol is dimerized into butanol.

9. Method according to claim 1 7, wherein in step b) acetaldehyde is dimerized into crotonaldehyde.

10. Method according to claim 9, wherein crotonaldehyde is reduced to 1-butanol.

11. Method according to claim 9, wherein crotonaldehyde is reduced to butyraldehyde.

12. Method according to claim 1, wherein in step c) an oxidation into butyric acid takes place.

13. Method according to claim 1, wherein in step c) a reduction into 1-butanol or an oxidation into butyraldehyde takes place.

14. Method according to claim 1, wherein in step c) 1-butanol or butyraldehyde is aminated reductively into 1-butylamines.

15. Method according to claim 9, wherein crotonaldehyde is converted into sorbic acid.

16. Method according to claim 9, wherein crotonaldehyde is converted into methoxybutanol.

17. Method according to claim 11, wherein said butyraldehyde is further processed into 2-ethylhexanol.

18. Method according to claim 11, wherein said butyraldehyde is further processed into 2-ethylhexanoic acid.

19. Method according to claim 8 3, wherein said n-butanol is further processed into n-butyl acetate.

20. Method according to claim 9, wherein said crotonaldehyde is converted into crotonic acid.
